(19) United States
(12) Patent Application Publication
(10) Pub. No.: US 2007/0095615 A1
(43) Pub. Date: May 3, 2007

(54) Methods and Apparatus for Merchandising Articles of Clothing

(76) Inventor: Donald Spector, New York, NY (US)

Correspondence Address: Glen M. Diehl;DIEHL SERVILLA LLC, Suite 110, 77 Brant Avenue, Clark, NJ, 07066, US

(21) Appl. No.: 11/558,644

(22) Filed: Nov. 10, 2006

Related U.S. Application Data
(63) Continuation-in-part of application No. 11/128,886, filed on May 13, 2005.

(51) Int. Cl. A61F 13/15; G06F 17/00; G08B 13/14; G05B 19/00

(52) U.S. Cl. 186/52; 235/375; 340/572.1; 340/5.91

(57) ABSTRACT

A method and apparatus for retailing articles of clothing are disclosed. An article of clothing is provided with an electronic memory device. The electronic memory device can be an optical disk or a jump drive or the like. The device can also be an RFID tag. It contains information about how to accessorize or match the article of clothing with other articles of clothing. The information includes both general information and specific recommendations as to which additional articles of clothing to buy. Also, a kiosk-type structure can be provided in a retail outlet. The structure includes an input device, a display and memory. An information tag, such as a bar code, is provided on each article of clothing. When the identifier tag is input to the input device, the information concerning the information about how to accessorize the article of clothing with other articles of clothing is displayed on the display. Other information that has been described as being provided on the electronic device can also be stored in the memory in the structure and can be displayed on the display. Additionally, goods other than clothing that can be accessorized can be sold this way.

### STATEMENT OF RELATED CASES

This application is a continuation-in-part of U.S. patent application Ser. No. 11/467,315, filed Aug. 25, 2006, which is hereby incorporated by reference.

### BACKGROUND OF THE INVENTION

This invention relates to the merchandising of articles of clothing.

Many people do not know how to match various articles of clothing. For example, certain patterns and colors match, and to look good, a person needs to know how to match these various items of clothing. Accessorizing is important in looking good when dressing. For example, when a customer buys a striped charcoal suit, certain shirts, ties and even shoes look good with the suit. It would be very helpful to purchasers of articles of clothing if they could have a system and method that helps them accessorize and to buy matching associated articles of clothing. Such a system would also help retailers by assisting their customers and by helping their sales people to become more knowledgeable about the articles of clothing they are selling.

### SUMMARY OF THE INVENTION

The present invention provides improved methods and retail sales kits to merchandise articles of clothing. It assists customers who may be unaware how to best buy additional articles of clothing that will match the article of clothing being purchased—that is how to accessorize. It will also assist manufacturers of clothing because they can assist their customers by providing information about the articles of clothing being sold and about the manufacturer. It will also assist retailers who can provide additional information about their goods, locations and the like, and can also increase the information being provided to their customers.

In accordance with one aspect of the present invention, a retail sales kit is provided. The retail sales kit includes an article of clothing and an electronic memory device. The electronic memory device contains information about how to accessorize the article of clothing with other articles of clothing. The electronic memory device is included with the article of clothing and is preferably attached to the article of clothing.

In one embodiment of the present invention, the electronic memory device is an optical disk. For example, a CD or a DVD can be used. If a CD is used, any of the available sized CDs can be used. Other forms of electronic memory devices can also be used. By way of example only, the electronic memory device can be a jump drive.

In another aspect of the present invention, a cover for the electronic memory device is included with the retail sales kit. The electronic memory device is placed in the cover, and the cover for the electronic memory device is attached to the article of clothing.

The information about how to accessorize the article of clothing with other articles of clothing preferably includes text information and color pictures. Additionally, information concerning a manufacturing company that makes the article of clothing can be stored on the electronic memory device. Further, information concerning a retail company that is selling the article of clothing can be stored on the electronic memory device. Also, information concerning how to dress in general can be stored on the electronic memory device.

In accordance with a further aspect of the present invention, an identifier tag is associated with the article of clothing. A structure having a display and an input device is provided in the retail outlet. The identifier tag can be a bar code or other information bearing device and the input device on the structure can be a bar code scanner or other input device. The information concerning how to accessorize the article of clothing with other articles of clothing is preferably stored in a memory in the structure. When the identifier tag is input to the input device, the information concerning the information about how to accessorize the article of clothing with other articles of clothing is displayed on the display. Other information that has been described as being provided on the electronic memory device can also be stored in the memory in the structure and can be displayed on the display. The structure also can include a processing circuit or processor and a connection to the Internet.

In accordance with another aspect of the present invention, a method of selling an article of clothing is provided. The method includes the steps of storing information concerning how to accessorize the article of clothing with other articles of clothing on an electronic memory device, covering the electronic memory device and the attaching the electronic memory device to the article of clothing.

The method further includes the step of placing the article of clothing and the electronic memory device on a sales rack and offering the combination of the article of clothing and the electronic memory device for sale.

In performing this method, all of the previously mentioned information can be stored on the electronic memory device.

In accordance with further aspects of the present invention, a retail sales kit that can communicate with a video or audio device is provided. The kit includes an article for sale and a device connected to the article for sale, the device having a memory and an interface circuit to the video or audio device.

The article for sale can be, for example, an article of clothing. By way of further example, it can be a shirt, pants, tie, etc.

The device connected to the article for sale can also have a processor. The video or audio device can be a personal computer, a radio, a portable computing device, a kiosk or any other device that can provide video or audio information.

In accordance with a further aspect of the invention, the interface circuit provides an interface to the video or audio device via a serial port, via a parallel port, via a USB port, via a PCI port, via a wireless interface or by any other available communication port.

The interface circuit can be a PCMCIA card. It can also be a Memory Stick. It can further be a wireless circuit. The interface circuit can also be embodied in an RFID tag, such as is commonly used to maintain inventories.

In accordance with further aspects of the present invention, the memory can store information relating to the subject matter of the article of clothing. The memory can also store an internet address of a site having information relating to the subject matter of the article for sale.

In accordance with another aspect of the present invention, a potential buyer of the article for sale is charged for accessing the information relating to the subject matter of the article for sale.

The memory can also store a key that allows access to the site.

As previously mentioned, the device can be an RFID tag. In this case, the interface circuit provides a wireless interface to the video or audio device. The RFID tag has a memory. The memory stores an identification of the article for sale. It also can store an address of an internet site. The internet site has information relating to the article for sale that can be viewed by a potential buyer. The memory can also store information related to the article for sale. In accordance with a further aspect of the present invention, the interface circuit in the RFID tag transmits the information related to the article to the video or audio device and the video or audio device plays the information to a potential buyer of the article for sale.

In accordance with another aspect of the present invention, a retail sales system including an internet site is provided. The system includes an article for sale and the article for sale having a code on it, wherein when the internet site is accessed with the code, information about the article for sale is provided. The code can be printed on the article for sale. Alternatively, wherein the code can be is stored on an electronic device attached to the article for sale.

The code can identify the article for sale to the internet site. The code can also provide access to the internet site.

In accordance with a further aspect of the present invention, a second code is provided on the article for sale. The second code can either be printed on the article for sale or stored on a device with memory that is attached to the article for sale. The code provides access to the internet site and the second code identifies the article for sale to the internet site.

The second code can be an inventory code that identifies the article for sale to an inventory system as well as to the internet site. In this embodiment of the present invention, it is preferred that the codes be stored on an RFID tag.

In accordance with yet another aspect of the present invention, where codes are wirelessly transmitted by the device of the present invention, an on/off switch is provided to control the wireless transmission of the codes. Alternatively, a cover can be provided over the device to prevent transmissions.

### DESCRIPTION OF THE DRAWINGS

FIG. 1 illustrates an article of clothing with an attached electronic memory device on a retail rack.

FIG. 2 illustrates optical disks of several sizes.

FIG. 3 illustrates a jump drive.

FIGS. 4 and 5 illustrate various organizations of the information on the electronic memory device and on the memory of the structure shown in FIGS. 6 and 7.

FIG. 6 illustrates a kiosk-type structure that is located in a retail outlet that is used in association with the articles of clothing or with identifying tags associated with the article of clothing.

FIG. 7 illustrates a block diagram of the electronics of the structure of FIG. 7.

FIGS. 8 and 9 illustrate further aspects of the present invention.

FIG. 10 illustrates a cover on a wireless device that is attached to an article for sale in accordance with a further aspect of the present invention.

### DESCRIPTION OF A PREFERRED EMBODIMENT

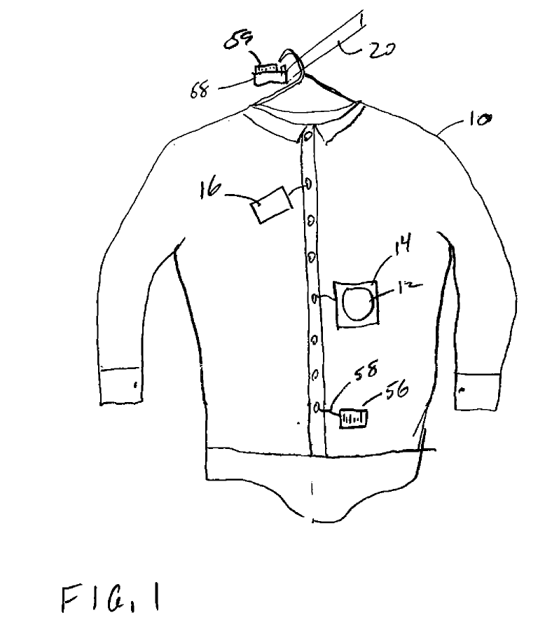

FIG. 1 illustrates an article of clothing 10 that will be offered for sale. The article of clothing is a shirt, although it can be any type of article of clothing, such as pants, a suit, a tie, a jacket, socks, or shoes. This list is provided without limitation. The article of clothing can also be a man's garment, a women's garment or a child's garment.

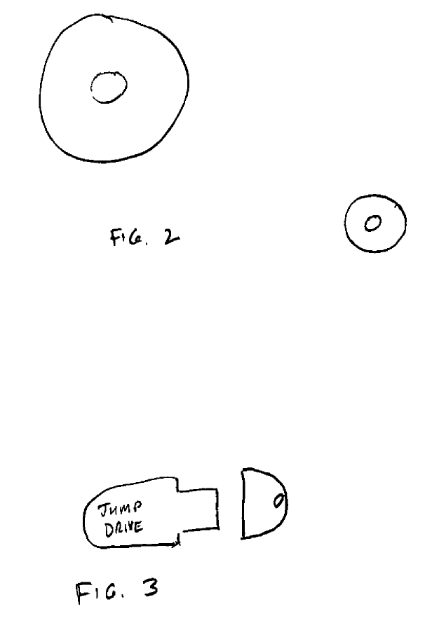

The shirt 10 has an electronic memory device 12 associated with it. The electronic memory device 12 is preferably an optical disk. The optical disk 12 is preferred because it can provide large amounts of information at a reasonable price. The optical disk 12 can be a CD. It can also be a DVD disk. The optical disk 12 can be any of the available sizes of disks. FIG. 2 illustrates two optical disks of different sizes that can be used.

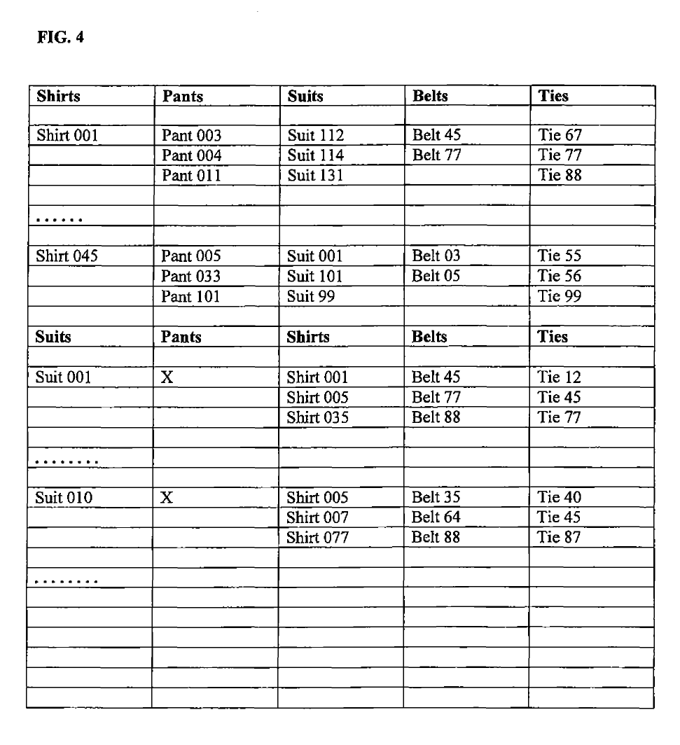

The electronic memory device 12 can also be a jump drive. FIG. 3 illustrates a jump drive. Such devices are available from Lexar and from SanDisk. Other electronic memory devices can also be used. For example, a memory stick, generally available from Sony could also be used. Older technology, such as a floppy disk could also be used, but is not preferred.

The electronic memory device 12 illustrated in FIG. 1 is covered with a cover 14. The cover is preferably a plastic sleeve, but any other of the well known enclosures can also be used.

The shirt also includes the typical tags 16 found on merchandise for sale in retail outlets. These tags include information about the article of clothing being offered for sale. A price sticker is also frequently found on these tags. The shirt 10 is shown hung on a retail rack 20.

The electronic memory device 12 stores information that will help a customer who is considering the purchase of the shirt 10 understand what other articles of clothing would be a match with the shirt. In short, the information on the electronic memory device 12 helps a customer accessorize the shirt 10 or any other article of clothing that the customer is considering purchasing.

The information on the electronic memory device 12 includes information about how to accessorize an article of clothing. Thus, the information tells a customer what other clothes to buy with a particular article of clothing. The information can provide specific recommendations on other articles of clothing to buy. That is, if a customer is considering a certain suit, the information can provide a recommendation on certain shirts and ties that match the suit to consider buying.

The information on the device 12 can also provide general recommendations on other articles of clothing to buy. So if a customer is considering a certain suit, say a blue striped suit, the information can provide a recommended list of types of shirts and ties to buy. For example, the information might recommend a white or light blue solid shirt and a dark blue tie.

The information on the device 12 can include text information and pictures. The text information describes the article of clothing being purchased as well as the articles of clothing that would match the item being purchased. The picture information could show how certain items would look together.

The information provided can also include information concerning a manufacturing company that makes the article of clothing is stored on the electronic memory device. It can also include information about a new technology or innovation that is used to manufacture the article of clothing. It can also include information concerning a retail company that is selling the article of clothing.

The information stored on the electronic memory device 12 can also include information concerning how to dress in general. For example, a well known book on how to dress could be provided in electronic format.

The information stored on the electronic memory device 12 can also include recommended outfits. So, for example, a suit, shirt, belt etc. can be recommended.

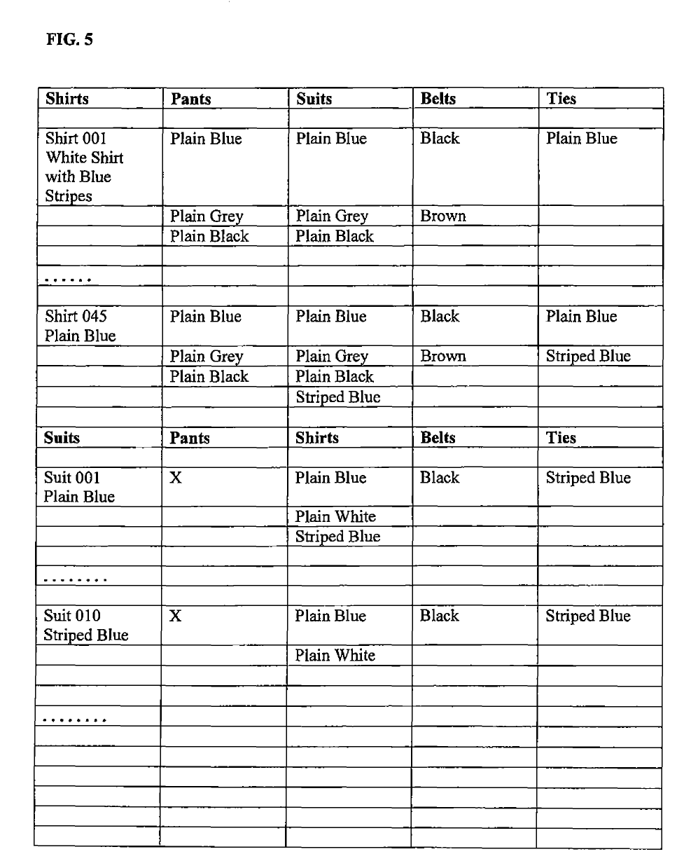

FIG. 4 illustrates one way to format information on the electronic memory device 12 and one type of information. In this organization, specific recommendations for items of clothing are provided.

In general, in FIG. 4, each shirt on sale in a retail store is included and for each shirt, there is one or more recommendations for pants, suits, belts and ties. Other items could also be provided. As an example, if a customer bought shirt 001, the customer could insert the electronic memory device 12 into a computer. The electronic memory device 12 would store a program that causes the information associated with the shirt 001 to be selectively displayed. The program would also allow the other information stored on the electronic memory device 12 to be selectively displayed as well, so the customer can view information about the manufacturer and the retailer.

Referring to FIG. 4, it can be seen that Pant 003, Pant 004 and Pant 011 are recommended matches for Shirt 001. Similarly, Suit 112, Suit 114 and Suit 131 are also recommended matches for Shirt 001. Recommendations for belts and ties are also made. These recommendations are made for all shirts in the retail store, Shirt 001-Shirt 045. Then specific recommendations for suits are made. It is preferred that specific recommendations be made for each item of clothing for sales or at least for most items for sale. Again, pictures showing the recommended item and/or the combination of the items can also be stored and displayed to the customer.

All of the information illustrated in FIG. 4 can be stored on the electronic memory device 12. Alternatively, only the information related to the article of clothing 10 needs to be stored on the electronic memory device 12.

FIG. 5 illustrates information stored on the electronic memory device 12 where general recommendations are made to a customer. Once again, each or most items offered for sale by a retail outlet are included. This time, the recommendations are general in nature. So, for Shirt 101, plain blue pants, plain grey pants and plain black pants are recommended. As far as suits are concerned, plain blue suits, plain grey suits and plain black suits are recommended. Similarly, general recommendations for belts and ties are made. Related pictures can be provided as well.

Again, while FIG. 5 illustrates recommendations for a plurality of articles of clothing, the electronic memory device 12 only needs to store information relating to the article of clothing that is associated with the electronic memory device 12.

Of course, a combination of information can be provided on the electronic memory device 12. So, for example, specific recommendations and general recommendations for each item of clothing could be provided. In this case, the information from FIG. 4 and from FIG. 5 is combined.

As previously mentioned, pictures can be associated with each recommended article of clothing to show how the combination of clothes would look.

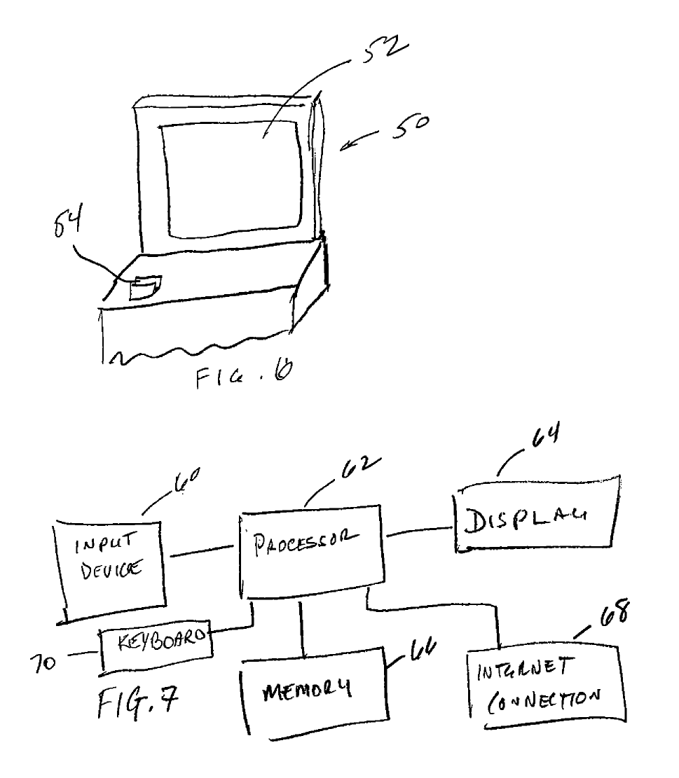

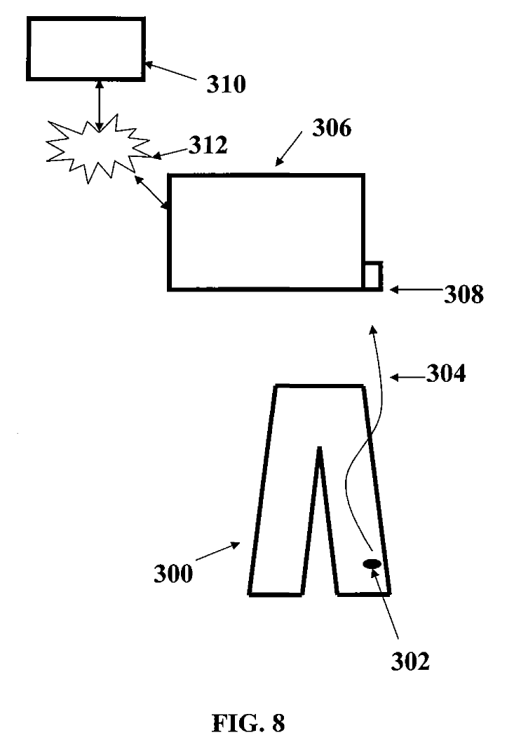

FIGS. 6 and 7 illustrate a kiosk-type structure 50 that can be placed in a retail outlet. The structure 50 is preferably on a stand so that a customer can walk up to the structure and interface with the structure 50. A customer using the structure 50 sees a display 52 and an input device 54. The customer can use the structure 50 to determine what other articles of clothing in the store would match or accessorize an article of clothing of interest to the customer while in the retail store. The structure 50 thereby helps the customer to make additional purchases to accessorize the article of clothing of interest while in a store instead of having to wait until they got home.

Referring back to FIG. 1, a tag 56 is preferably attached to the article of clothing 10. The tag 56 is attached to the article of clothing 10, preferably via a plastic string 58 in a well known manner. The tag 56 includes a bar code, or other identifying information, to indicate the identity of the article of clothing.

To use the structure 50, the customer scans the tag 56 through the input device 54. The input device 54 is a bar code scanner or other scanning device that can read the information on the tag 56. The input device 54 identifies the article of clothing of interest. Based on the identity of the article of clothing, the structure 50 specifies other articles of clothing that will match or go with the article of clothing of interest.

Referring back to FIG. 1 again, the retail display of the article of clothing 10 can also include a holder 58. The holder 58 contains one or more identifying cards 59. The cards 60 provide the same function as the tag 56. Thus, the cards 59 can have a bar code that identifies the article of clothing 10. A customer can take one of the cards 59 to the kiosk-type structure 50 to find other articles of clothing that match the article of clothing 10. Thus, the card 59 performs the same function as the tag 56.

FIG. 5 illustrates a block diagram of the electronics of the structure 50, which could simply be a personal computer programmed to perform in a manner described herein. The structure includes an input device 60, a processor 62, a display 64, a memory 66 and an internet connection device 68. The input device 60, such as a bar code scanner, is connected to the processor 62. The processor 62 can be a microprocessor or a simple processing circuit or even a simple processing device. The processor 62 is connected to the display 64, to the memory 66 and to the internet connection device 68.

When the tag 56 or the card 59 is swiped or otherwise input to the input device 60, the input device generates a signal that identifies the article of clothing of interest. This identifying signal is input to the processor 62. The processor 62 uses the identifying signal to access the memory 64. The memory 64 stores suggestions of specific articles of clothing that would match the article of clothing of interest, such as the shirt 10. Thus, for example, the memory 64 could store an identifier of the shirt 10 and suggested other items to buy that match the shirt 10, such as suits, pants, belts, ties, etc. Of course, the memory 64 would store suggested other items to buy for all of the items for sale in the store, including pants, suits, etc. Thus, there would be a listing for every item or at least most items in the retail store. The information in the memory 64 could be organized in a manner similar to that illustrated in FIG. 4. In this case, however, all of the information in FIG. 4 is stored in the memory 64. Preferably, information about all of the items for sale in a retail store is provided.

Alternatively, the memory 64 could store descriptions of other clothing items that would match the article of clothing of interest. Thus, for example, if the shirt 10 is blue and striped, then a description of matching items that specifies colors and designs could be provided in the memory 64 in association with the identified article of clothing of interest. An example of the organization of the memory 64 in this embodiment is illustrated in FIG. 5. Again, information about all of the items for sale in a retail store is provided.

After the customer inputs the tag 56 or the card 59, the processor 62 determines the article of clothing from the tag 56 or card 59 and accesses the memory 64 with that information. The information retrieved from the memory 64 is formatted for display by the processor 62. The processor 62 causes that information to be displayed to a customer on the display 62.

The internet connection 68 is provided to allow a manufacturer of clothing to download information relating to the manufacturer's clothing that is on sale in the store. The processor 62 reads the information relating to each item offered by the manufacturer and stores that information in the memory 64. Additionally, a keyboard 70 is provided to allow a retail store to enter its own information concerning other items of clothing that are on sale in the store. The retail store owner can also enter information about the retail store and its merchandise, including any specials that the retail store may be offering.

The present invention has been described with respect to clothing and accessorizing. The method and apparatus disclosed herein can also be used with the merchandising of other retail products that might require additional add on products (such as computers, mobile sound equipment, cars etc.) as well as general instructions on how to use the product. For example, the present invention could also be used with pharmaceuticals to provide information and visual pictures of contra indications that can be sent to both doctors and patients. This could be part of a normal medication practice in the future.

It is also an aspect of the present invention to provide a kiosk-like structure in a mall where customers could enter information concerning a product to check the availability of the product at various stores in the mall or in a surrounding area or on the Internet and to check the pricing of the product from these locations. Alternatively, a call center could be provided wherein a customer can call—via a land line or cell phone—to enter the product number and to check availability and pricing from a variety of locations. Further, if the consumer enters the price of the product along with the product identification into the kiosk-like structure in the mall or during the call to the call center, either of these devices could provide retail outlets that have the product available for less money.

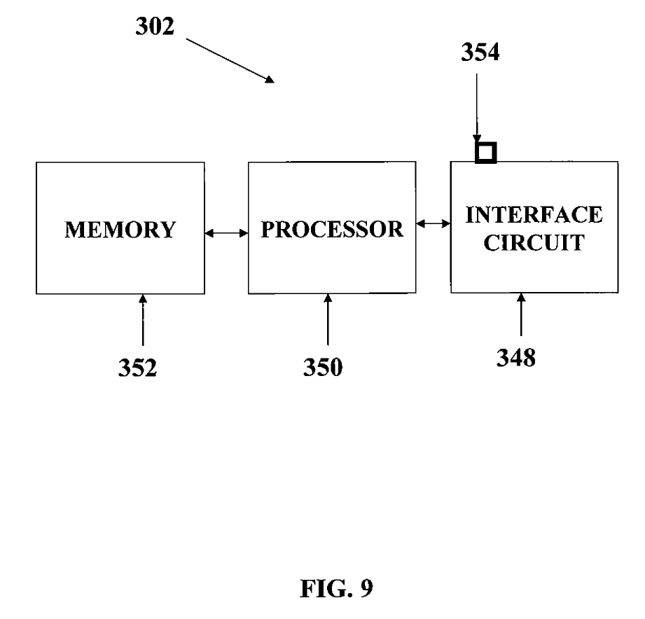

FIGS. 8 and 9 illustrate further aspects of the present invention. Referring to FIG. 13, an article for sale 300, such as pants, has an RFID tag 302 on it. The RFID tag 302 is shown on the front of the pants 300 in FIG. 8, however, the RFID tag 302 can be located anywhere on the pants 300.

RFID tags 302 typically communicate wirelessly and can be used, for example, to maintain inventories. Referring to FIG. 8, the RFID tag 302 sends a wireless signal 304 to a video or audio device 306. The video or audio device 306 receives the signal via a wireless interface 308. The video or audio device 306 can be any of the previously listed deices.

Referring to FIG. 9, the RFID tag 302 has a memory on it. It also has a wireless interface circuit 348. It can also optionally have a processor 350.

The memory on the RFID tag is typically small. In accordance with one aspect of the present invention, the memory stores an identification of the article for sale. In accordance with a further aspect of the present invention, the memory can also store an address of an internet site. In accordance with yet another aspect of the present invention, the memory can also store information related to the article for sale. The memory on the RFID tag 302 can store any combination of the above information.

In accordance with another aspect of the invention, the video or audio device can access an internet site 310 via the internet 312. The internet site has information related to the book 300 that can be accessed by the video or audio device 306 and displayed to a potential buyer of the article for sale. The video or audio device 306 is preferably programmed to read the signal 304 from the RFID tag 302, obtain the address of the site 310, and either provide access to the site 310 or automatically download information from the site 310 to the device 306. That information can also be displayed to a potential buyer.

The information from the memory 352 can be transmitted wirelessly to the device 306 and displayed on the device 306 to a potential buyer. The device 306 can be, for example, a kiosk. The information can include price, available colors, styling choices, critics reviews, etc.

Devices other than a RFID tag can be used. The device will typically include a memory 352 and an interface circuit 348, and will optionally include a processor 350, as shown in FIG. 9. For example, the device can be a specially designed circuit, or any of the previously mentioned devices can also be used.

The type of interface circuit provided depends on the type of connection to the device 306. The processor 350 is optional. If a passive interface to the device 306 that does not require processor control is used, then a processor 350 is not needed. Any type of memory 350, including DRAM, SRAM, EPROM, PROM, Masked ROM, NVRAM, Flash or EEPROM, can be used.

The interface circuit 348 can be an off-the-shelf integrated circuit device specially designed for the port on the device 306. Alternatively, it can also be a specially designed circuit made from discrete components, ASICs or the like. The same is true of the processor 350.

The interface circuit 348 can also include a serial port, a parallel port, a USB port, a PCI port. The interface circuit 348 can also provide a wireless interface to the device 306. For example, the device 306 can provide a wireless USB (“WUSB”) connection to the device 306. Any other available wireless interface can also be used.

In accordance with further aspects of the present invention, the device 302 can be a PCMCIA card or a Memory Stick, or any other type of standard memory card.

The memory 350 in any of these devices can store the information previously described.

A potential buyer of the article for sale can be optionally charged for information downloaded in accordance with the teachings of the present invention.

In accordance with another aspect of the present application, any kind of alpha numeric code can be either printed somewhere on an article for sale or on a tag on the article for sale, such as a hang tag, that can be fed into an electronic device capable of interacting with the Internet in which these alphanumeric numbers are used to open a site that is specifically geared to the informational content associated with the printed material. These codes can be either in print, stored on a CD-ROM or stored on other devices capable of storing such a number.

It is believed that the wireless operation provides a convenience to the consumer, however, it is possible that an “unlocking key” of alpha numeric numbers can be punched into an Ipod, Zune, cellular phone, laptop computer or regular computer and open up a site that could then be used to provide supplemental material either free or for an up charge. This “key” could also be used to communicate with a server that communicates with another site (such as an academic institution) to provide CE credit.

It is believed that in the future the wireless systems, the plug in systems and the code systems will all be used in the addition of supplemental material to printed matter. Even radio satellite systems may have the ability to address specific transmissions through satellite radio if a code is given to them that activates a dedicated web site that can then provide a variety of multimedia content.

In accordance with one aspect of the present invention, a plurality of codes can be provided on the article for sale, either in printed format or stored on an electronic device with memory, such as an RFID tag. A first code can provide an address to an internet site and/or a key to the internet site. The internet site has information related to the article for sale that can be viewed or heard. A second code can provide an identification of the article for sale.

The codes can be supplied to a device 306 via any of the discussed methods, including, for example, by wireless communications. The device 306 uses the first code to access an internet site 310. The code can either be an address and/or a key to the site. The second code is transmitted to the internet site 310 once communications with the site 310 are established. The internet site 310 then uses the second code that identifies the article for sale to access information relating to the article for sale. The internet site 310 then sends the information relating to the article for sale to the device 306 so that the information can be displayed to a potential buyer of the article for sale.

Referring to FIG. 9, an on/off switch 354 on the interface circuit 348 is optionally provided. When an RFID device, or any other wireless transmission device, is used in accordance with the teachings of the present invention, there is a possible bottleneck at the computer 306. If a number of articles for sale approach the computer 306 to transmit codes to the computer 306, the computer 306 could be overloaded. It is therefore preferable to be able to selectively enable and disable the wireless transmission of the interface circuit 348. The on/off switch 354 selectively enables or disables the interface circuit 348. Thus, a potential purchaser of an article for sale can selectively enable the wireless transmission from the device 306 when nearing the computer 306.

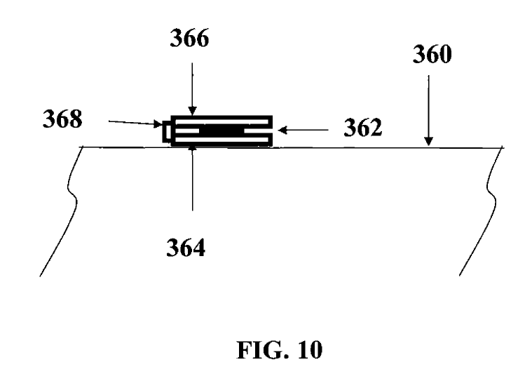

Referring to FIG. 10, a cover 366 can be used to selectively enable and disable the wireless transmissions from the RFID tag 362. The RFID tag 362 sits in a bottom receptacle 364. The bottom receptacle 364 is secured to the article for sale 360. A cover 366 is secured to the bottom receptacle 364 by a flap 368. When the cover 366 is over the RFID tag 362, wireless transmission to the computer 306 is prevented.

Although the invention herein has been described with reference to particular embodiments, it is to be understood that these embodiments are merely illustrative of the principles and applications of the present invention. It will be apparent to those skilled in the art that various modifications and variations can be made in the apparatus and methods of the present invention without departing from the spirit and scope of the invention. Thus, it is intended that the present invention include modifications and variations that are within the scope of the appended claims and their equivalents.

1. A retail sales kit that can communicate with a video or audio device, comprising:
an article for sale; and
a device connected to the article for sale, the device having a memory and an interface circuit to the video or audio device.

2. The retail sales kit of claim 1, wherein the article for sale is clothing.

3. The retail sales kit of claim 1, the device further having a processor.

4. The retail sales kit of claim 1, wherein the video or audio device is a personal computer.

5. The retail sales kit of claim 1, wherein the video or audio device is a radio.

6. The retail sales kit of claim 1, wherein the video or audio device is a portable computing device.

7. The retail sales kit of claim 1, wherein the interface circuit provides an interface to the video or audio device via a serial port.

8. The retail sales kit of claim 1, wherein the interface circuit provides an interface to the video or audio device via a parallel port.

9. The retail sales kit of claim 1, wherein the interface circuit provides an interface to the video or audio device via a USB port.

10. The retail sales kit of claim 1, wherein the interface circuit provides an interface to the video or audio device via a PCI port.

11. The retail sales kit of claim 1, wherein the interface circuit provides an interface to the video or audio device via a wireless interface.

12. The retail sales kit of claim 1, wherein the interface circuit is a PCMCIA card.

13. The retail sales kit of claim 1, wherein the interface circuit is a Memory Stick.

14. The retail sales kit of claim 1, wherein the interface circuit is a wireless circuit.

15. The retail sales kit of claim 1, wherein the memory stores information relating to the subject matter of the article for sale.

16. The retail sales kit of claim 1, wherein the memory stores an internet address of a site having information relating to the subject matter of the article for sale.

17. The retail sales kit of claim 16, wherein a user of the article is charged for accessing the information relating to the subject matter of the article for sale.

18. The retail sales kit of claim 16, wherein the memory stores a key that allows access to the site.

19. The retail sales kit of claim 1, wherein the device is an RFID tag.

20. The retail sales kit of claim 19, wherein the interface circuit provides a wireless interface to the video or audio device.

21. The retail sales kit of claim 20, wherein the memory stores an identification of the article for sale.

22. The retail sales kit of claim 20 wherein the memory stores an address of an internet site.

23. The retail sales kit of claim 20, wherein the memory stores information related to the article for sale.

24. The retail sales kit of claim 23, wherein the interface circuit transmits the information related to the article to the video or audio device and the video or audio device plays the information.

25. The retail sales kit of claim 23, wherein the memory stores information related to the article for sale.

26. The retail sales kit of claim 23, wherein the video or audio device is programmed to access the internet site and the internet site has information related to the printed publication that can be accessed by the video or audio device.

27. The retail sales kit of claim 11, further comprising means for disabling the wireless interface.

28. The retail sales kit of claim 11, further comprising an on/off switch on the interface circuit.

29. The retail sales kit of claim 1, further comprising a cover that can disable the interface circuit.

30. A retail sales system including an internet site, comprising:
an article for sale;
the article for sale having a code on it;
wherein when the internet site is accessed with the code, information about the article for sale is provided.

31. The retail sales system of claim 30, wherein the code is printed on the article for sale.

32. The retail sales system of claim 30, wherein the code is stored on an electronic device attached to the article for sale.

33. The retail sales system of claim 30, wherein the code identifies the article for sale to the internet site.

34. The retail sales system of claim 30, wherein the code provides access to the internet site.

35. The retail sales system of claim 30, comprising a second code on the article for sale.

36. The retail sales system of claim 35, wherein the code provides access to the internet site and the second code identifies the article for sale to the internet site.

37. The retail sales system of claim 36, wherein the code and the second code is stored on an RFID tag.

38. The retails sales system of claim 37, wherein the second code is an inventory code that is used in an inventory control system.
